(19) United States
(12) Patent Application Publication
(10) Pub. No.: US 2012/0238876 A1
(43) Pub. Date: Sep. 20, 2012

(54) ULTRASOUND DIAGNOSTIC APPARATUS AND METHOD OF PRODUCING ULTRASOUND IMAGE

(75) Inventors: Tsuyoshi TANABE, Kanagawa (JP); Yuji OHSHIMA, Kanagawa (JP)

(73) Assignee: FUJIFILM CORPORATION, Tokyo (JP)

(21) Appl. No.: 13/313,647

(22) Filed: Dec. 7, 2011

(30) Foreign Application Priority Data
Mar. 18, 2011 (JP) 2011-060911
Mar. 18, 2011 (JP) 2011-060956
Mar. 18, 2011 (JP) 2011-061035

(51) Int. Cl. A61B 8/14

(52) U.S. Cl. 600/444

(57) ABSTRACT

An ultrasound diagnostic apparatus includes: an ultrasound probe which has a one-dimensional array-type transducer array and an array moving unit moving the transducer array in a direction substantially orthogonal to the array direction of the transducer array; a transmission and reception circuit which electronically scans the transducer array, and transmits and receives an ultrasonic beam toward a subject to acquire two-dimensional image data; and a controller which, when the internal temperature of the ultrasound probe is equal to or higher than a first set value, controls the transmission and reception circuit such that the transmission and reception or the reception of an ultrasonic beam for at least a part of a region other than a region of interest is paused.

### BACKGROUND OF THE INVENTION

The present invention relates to an ultrasound diagnostic apparatus and a method of producing an ultrasound image, and in particular, to an ultrasound diagnostic apparatus which combines electronic scan and mechanical scan of a transducer array to produce a three-dimensional ultrasound image.

An ultrasound diagnostic apparatus using an ultrasound image has hitherto been put into practical use in the field of medicine. In general, this kind of ultrasound diagnostic apparatus has an ultrasound probe embedded with a transducer array and an apparatus body connected to the ultrasound probe. An ultrasonic wave is transmitted from the ultrasound probe toward a subject, an ultrasonic echo from the subject is received by the ultrasound probe, and the reception signal is electrically processed in the apparatus body to produce an ultrasound image.

A transducer array having a plurality of ultrasound transducers arranged one-dimensionally is widely used. The transducer array is electronically scanned to obtain a two-dimensional tomographic image. When seeing an image in a vertical direction with respect to the tomographic image, that is, an image in front of or behind the tomographic image, the position or angle of the ultrasound probe is changed to produce different tomographic images. However, it is necessary to produce a large number of two-dimensional tomographic images depending on the shape, size, or the like of a site under diagnosis to recognize the situation of the site under diagnosis, and a sense of discomfort may be given to a patient at the time of movement of the ultrasound probe.

Accordingly, JP 2009-240525 A describes an ultrasound diagnostic apparatus in which a transducer array is electronically scanned to acquire two-dimensional image data and the transducer array is also mechanically scanned in a direction substantially orthogonal to the array direction of the transducer array, thereby producing a three-dimensional ultrasound image. According to this ultrasound diagnostic apparatus, it becomes possible to produce a three-dimensional ultrasound image without moving an ultrasound probe.

However, in the ultrasound probe of such an ultrasound diagnostic apparatus, a scan mechanism which mechanically scans with the transducer array is accommodated in the housing of the probe, and when diagnosis is performed, heat is generated from the transducer array and the scan mechanism, causing a rise in the temperature of the housing of the ultrasound probe.

In particular, an ultrasound diagnostic apparatus is known in which a circuit board for signal processing is embedded in the ultrasound probe, and a reception signal output from the transducer array is subjected to digital processing and then transmitted to the apparatus body through wireless communication or wired communication, thereby reducing the influence of noise and obtaining a high-quality ultrasound image. In this ultrasound diagnostic apparatus, heat is generated from the circuit board, and a rise in the temperature of the housing is caused. If the temperature of the housing increases, it becomes difficult to assure a stable operation of each circuit in the ultrasound probe.

### SUMMARY OF THE INVENTION

The invention has been finalized in order to solve the drawbacks inherent in the related art, and an object of the invention is to provide an ultrasound diagnostic apparatus and a method of producing an ultrasound image capable of obtaining a high-quality three-dimensional ultrasound image while suppressing a rise in the internal temperature of an ultrasound probe.

An ultrasound diagnostic apparatus according to a first aspect of the invention includes

an ultrasound probe which has a one-dimensional array-type transducer array and an array moving unit moving the transducer array in a direction substantially orthogonal to the array direction of the transducer array,

a transmission and reception circuit which electronically scans with the transducer array, and transmits and receives an ultrasonic beam toward a subject to acquire two-dimensional image data,

an image producer which produces a three-dimensional ultrasound image using two-dimensional image data acquired by the transmission and reception circuit while mechanically scanning with the transducer array in a direction substantially orthogonal to the array direction of the transducer array by the array moving unit,

a region of interest setter which sets a region of interest in an imaging region,

a temperature sensor which detects an internal temperature of the ultrasound probe, and

a controller which, when the internal temperature of the ultrasound probe detected by the temperature sensor is equal to or higher than a first set value, controls the transmission and reception circuit such that the transmission and reception or the reception of an ultrasonic beam for at least a part of a region other than the region of interest set by the region of interest setter is paused.

An ultrasound diagnostic apparatus according to a second aspect of the invention includes

an ultrasound probe which has a one-dimensional array-type transducer array and an array moving unit moving the transducer array in a direction substantially orthogonal to the array direction of the transducer array,

a transmission and reception circuit which electronically scans with the transducer array, and transmits and receives an ultrasonic beam toward a subject to acquire two-dimensional image data,

an image producer which produces a three-dimensional ultrasound image using two-dimensional image data acquired by the transmission and reception circuit while mechanically scanning with the transducer array in a direction substantially orthogonal to the array direction of the transducer array by the array moving unit,

a region of interest setter which sets a region of interest in an imaging region,

a temperature sensor which detects an internal temperature of the ultrasound probe, and

a controller which, when the internal temperature of the ultrasound probe detected by the temperature sensor is equal to or higher than a first set value, controls the transmission and reception circuit such that the transmission and reception or the reception of an ultrasonic beam for at least a part of a region other than the region of interest set by the region of interest setter is intermittently performed.

An ultrasound diagnostic apparatus according to a third aspect of the invention includes

an ultrasound probe which has a one-dimensional array-type transducer array and an array moving unit moving the transducer array in a direction substantially orthogonal to the array direction of the transducer array,

a transmission and reception circuit which electronically scans with the transducer array, and transmits and receives an ultrasonic beam toward a subject to acquire two-dimensional image data,

an image producer which produces a three-dimensional ultrasound image using two-dimensional image data acquired by the transmission and reception circuit while mechanically scanning with the transducer array in a direction substantially orthogonal to the array direction of the transducer array by the array moving unit,

a region of interest setter which sets a region of interest in an imaging region,

a temperature sensor which detects an internal temperature of the ultrasound probe, and

a controller which, when the internal temperature of the ultrasound probe detected by the temperature sensor is equal to or higher than a first set value, controls the transmission and reception circuit such that the transmission and reception or the reception of an ultrasonic beam for at least a part of a region other than the region of interest set by the region of interest setter is performed with decreased spatial resolution.

A method of producing an ultrasound image according to a fourth aspect of the invention includes the steps of

electronically scanning with a one-dimensional array-type transducer array of an ultrasound probe by a transmission and reception circuit and transmitting and receiving an ultrasonic beam toward a subject to acquire two-dimensional image data, and mechanically scanning with the transducer array in a direction substantially orthogonal to the array direction of the transducer array to acquire a plurality of pieces of two-dimensional image data,

producing a three-dimensional ultrasound image using a plurality of pieces of acquired two-dimensional image data,

setting a region of interest in an imaging region,

detecting an internal temperature of the ultrasound probe, and

when the detected internal temperature of the ultrasound probe is equal to or higher than a first set value, controlling the transmission and reception circuit such that the transmission and reception or the reception of an ultrasonic beam for at least a part of a region other than the region of interest is paused.

A method of producing an ultrasound image according to a fifth aspect of the invention includes the steps of

electronically scanning with a one-dimensional array-type transducer array of an ultrasound probe by a transmission and reception circuit and transmitting and receiving an ultrasonic beam toward a subject to acquire two-dimensional image data, and mechanically scanning with the transducer array in a direction substantially orthogonal to the array direction of the transducer array to acquire a plurality of pieces of two-dimensional image data,

producing a three-dimensional ultrasound image using a plurality of pieces of acquired two-dimensional image data,

setting a region of interest in an imaging region,

detecting an internal temperature of the ultrasound probe, and

when the detected internal temperature of the ultrasound probe is equal to or higher than a first set value, controlling the transmission and reception circuit such that the transmission and reception or the reception of an ultrasonic beam for at least a part of a region other than the region of interest is intermittently performed.

A method of producing an ultrasound image according to a sixth aspect of the invention includes the steps of

electronically scanning with a one-dimensional array-type transducer array of an ultrasound probe by a transmission and reception circuit and transmitting and receiving an ultrasonic beam toward a subject to acquire two-dimensional image data, and mechanically scanning with the transducer array in a direction substantially orthogonal to the array direction of the transducer array to acquire a plurality of pieces of two-dimensional image data,

producing a three-dimensional ultrasound image using a plurality of pieces of acquired two-dimensional image data,

setting a region of interest in an imaging region,

detecting an internal temperature of the ultrasound probe, and

when the detected internal temperature of the ultrasound probe is equal to or higher than a first set value, controlling the transmission and reception circuit such that the transmission and reception or the reception of an ultrasonic beam for at least a part of a region other than the region of interest is performed with decreased spatial resolution.

### BRIEF DESCRIPTION OF THE DRAWINGS

FIG. 1 is a block diagram showing the configuration of an ultrasound diagnostic apparatus according to Embodiment 1 of the invention.

FIG. 2 is a flowchart showing the operation of Embodiment 1.

FIG. 3 is a diagram showing a scan method of a transducer array in a normal state in Embodiment 1.

FIG. 4 is a diagram showing a scan method of the transducer array when the internal temperature of an ultrasound probe is equal to or higher than a first set value in Embodiment 1.

FIG. 5 is a diagram showing a scan method of a transducer array when the internal temperature of an ultrasound probe is equal to or higher than a second set value in Embodiment 2.

FIG. 6 is a diagram showing a scan method of the transducer array when the internal temperature of an ultrasound probe is equal to or higher than a third set value in Embodiment 2.

FIG. 7 is a diagram showing a scan method of a transducer array when the internal temperature of an ultrasound probe is equal to or higher than a third set value in a modification of Embodiment 2.

FIG. 8 is a diagram showing a scan method of a transducer array when the internal temperature of an ultrasound probe is equal to or higher than a first set value in Embodiment 3.

FIG. 9 is a diagram showing a scan method of a transducer array when the internal temperature of an ultrasound probe is equal to or higher than a first set value in a modification of Embodiment 3.

FIG. 10 is a diagram showing a scan method of a transducer array when the internal temperature of an ultrasound probe is equal to or higher than a first set value in another modification of Embodiment 3.

FIG. 11 is a block diagram showing the configuration of an ultrasound diagnostic apparatus according to Embodiment 4.

FIG. 12 is a flowchart showing the operation of Embodiment 4.

FIG. 13 is a diagram showing a scan method of a transducer array when the internal temperature of an ultrasound probe is equal to or higher than a first set value in Embodiment 4.

FIG. 14 is a diagram showing a scan method of a transducer array when the internal temperature of an ultrasound probe is equal to or higher than a second set value in Embodiment 5.

FIG. 15 is a diagram showing a scan method of the transducer array when the internal temperature of an ultrasound probe is equal to or higher than a third set value in Embodiment 5.

FIG. 16 is a diagram showing a scan method of a transducer array when the internal temperature of an ultrasound probe is equal to or higher than a third set value in a modification of Embodiment 5.

FIG. 17 is a diagram showing a scan method of a transducer array when the internal temperature of an ultrasound probe is equal to or higher than a first set value in Embodiment 6.

FIG. 18 is a diagram showing a scan method of a transducer array when the internal temperature of an ultrasound probe is equal to or higher than a first set value in a modification of Embodiment 6.

FIG. 19 is a diagram showing a scan method of a transducer array when the internal temperature of an ultrasound probe is equal to or higher than a first set value in another modification of Embodiment 6.

FIG. 20 is a diagram showing a scan method of a transducer array in a normal state in Embodiment 7.

FIG. 21 is a diagram showing a scan method of the transducer array when the internal temperature of an ultrasound probe is equal to or higher than a first set value in Embodiment 7.

FIG. 22 is a diagram showing a scan method of a transducer array when the internal temperature of an ultrasound probe is equal to or higher than a second set value in Embodiment 8.

FIG. 23 is a diagram showing a scan method of the transducer array when the internal temperature of an ultrasound probe is equal to or higher than a third set value in Embodiment 8.

FIG. 24 is a diagram showing a scan method of a transducer array when the internal temperature of an ultrasound probe is equal to or higher than a third set value in a modification of Embodiment 8.

FIG. 25 is a diagram showing a scan method of a transducer array when the internal temperature of an ultrasound probe is equal to or higher than a first set value in Embodiment 9.

FIG. 26 is a diagram showing a scan method of a transducer array when the internal temperature of an ultrasound probe is equal to or higher than a first set value in modification of Embodiment 9.

FIG. 27 is a diagram showing a scan method of a transducer array when the internal temperature of an ultrasound probe is equal to or higher than a first set value in another modification of Embodiment 9.

### DETAILED DESCRIPTION OF THE INVENTION

Hereinafter, embodiments of the invention will be described on the basis of the accompanying drawings.

### Embodiment 1

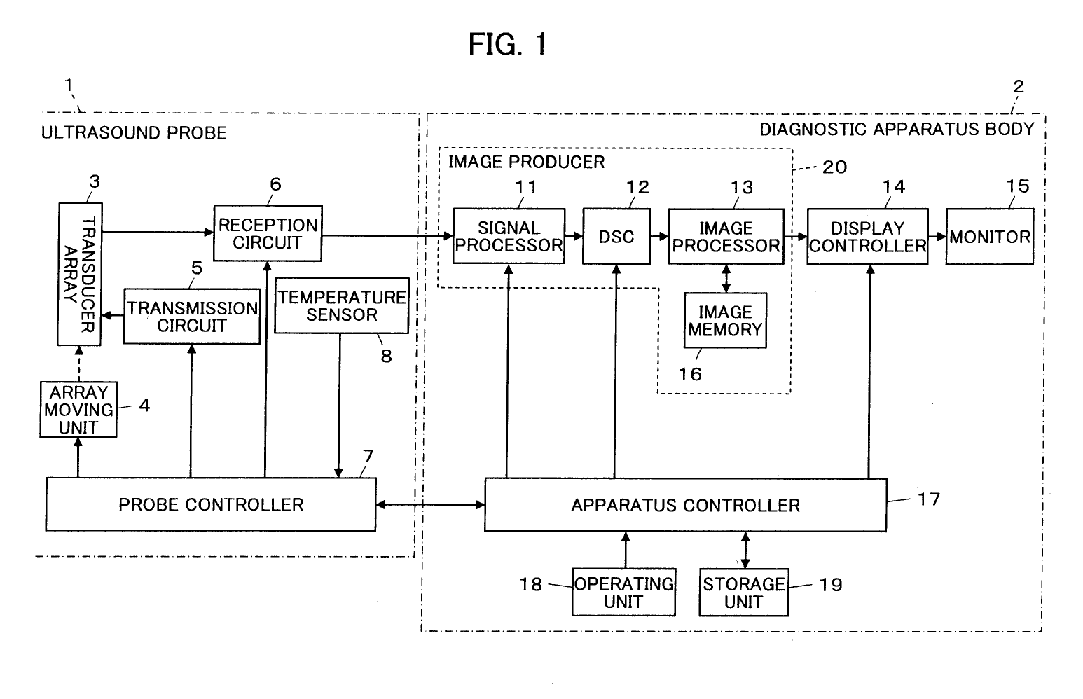

FIG. 1 shows the configuration of an ultrasound diagnostic apparatus according to Embodiment 1 of the invention. The ultrasound diagnostic apparatus includes an ultrasound probe 1, and a diagnostic apparatus body 2 connected to the ultrasound probe 1.

The ultrasound probe 1 has a transducer array 3 having a plurality of ultrasound transducers arranged one-dimensionally. An array moving unit 4 is connected to the transducer array 3, and a transmission circuit 5 and a reception circuit 6 are also connected to the transducer array 3. A probe controller 7 is connected to the array moving unit 4, the transmission circuit 5, and the reception circuit 6. A temperature sensor 8 which detects the internal temperature of the ultrasound probe 1 is embedded in the ultrasound probe 1, and the temperature sensor 8 is connected to the probe controller 7. The temperature sensor 8 is disposed, for example, in the vicinity of the reception circuit 6 where heat is expected to be generated, particularly, when the ultrasound diagnostic apparatus is in operation.

The diagnostic apparatus body 2 has a signal processor 11 connected to the reception circuit 6 of the ultrasound probe 1. A DSC (Digital Scan Converter) 12, an image processor 13, a display controller 14, and a monitor 15 are sequentially connected to the signal processor 11, and an image memory 16 is connected to the image processor 13. An apparatus controller 17 is connected to the signal processor 11, the DSC 12, and the display controller 14. An operating unit 18 and a storage unit 19 are connected to the apparatus controller 17.

The probe controller 7 of the ultrasound probe 1 and the apparatus controller 17 of the diagnostic apparatus body 2 are connected together.

The transducer array 3 of the ultrasound probe 1 has a plurality of ultrasound transducers arranged one-dimensionally. These ultrasound transducers are constituted by transducers in which electrodes are formed at both ends of a piezoelectric body made of piezoelectric ceramic represented by, for example, PZT (lead zirconate titanate), a polymer piezoelectric device represented by PVDF (polyvinylidene fluoride), or piezoelectric monocrystal represented by PMN-PT (lead magnesium niobate-lead titanate solid solution).

If a pulsed or continuous-wave voltage is applied to the electrodes of each transducer, the piezoelectric body expands and contracts, and pulsed or continuous-wave ultrasonic waves are generated from the transducers and synthesized to form an ultrasonic beam. When receiving propagating ultrasonic waves, the transducers expand and contract to generate electric signals, and the electric signals are output as reception signals of ultrasonic waves.

The transducer array 3 is disposed pivotably or slidably in a direction substantially orthogonal to the array direction of the ultrasound transducers, and is configured to repeatedly pivot in a predetermined period and angle range or to linearly reciprocate with a predetermined cycle and stroke by the actuation of the array moving unit 4. As the array moving unit 4, various motors, actuators, or the like may be used.

The transmission circuit 5 includes, for example, a plurality of pulsars. The transmission circuit 5 adjusts the delay amount of each actuation signal on the basis of a transmission delay pattern selected in response to a control signal from the probe controller 6 such that ultrasonic waves transmitted from a plurality of ultrasound transducers of the transducer array 3 form an ultrasonic beam, and supplies the adjusted delay amount to each of a plurality of ultrasound transducers.

The reception circuit 6 performs a reception focus process in which a reception signal transmitted from each ultrasound transducer of the transducer array 3 is amplified, subjected to A/D conversion, and reception signals are added with delay added thereto in accordance with a sound velocity or a sound velocity distribution set on the basis of a reception delay pattern selected in response to a control signal from the probe controller 6. With this reception focus process, the focus of an ultrasonic echo is narrowed to produce reception data (sound ray signal).

The transmission circuit 5 and the reception circuit 6 constitute a transmission and reception circuit of the invention.

The temperature sensor 8 detects the internal temperature Tp of the ultrasound probe 1 and outputs the result to the probe controller 7.

The probe controller 7 controls the respective units of the ultrasound probe 1 on the basis of various control signals transmitted from the apparatus controller 17 of the diagnostic apparatus body 2.

The signal processor 11 of the diagnostic apparatus body 2 corrects attenuation depending on the distance in accordance with the depth of the reflection position of the ultrasonic wave for reception data produced by the reception circuit 6 of the ultrasound probe 1, and performs an envelope detection process to produce a B-mode image signal which is tomographic image information relating to a tissue in the subject.

The DSC 12 converts (raster-converts) the B-mode image signal produced by the signal processor 11 to an image signal according to a normal television signal scan system.

The image processor 13 performs various necessary image processes, such as a gradation process, on the B-mode image signal input from the DSC 12 to produce two-dimensional image data, and stores two-dimensional image data in the image memory 16. Simultaneously, the image processor 13 produces three-dimensional image data from a plurality of pieces of two-dimensional image data stored in the image memory 16, and outputs three-dimensional image data to the display controller 14.

The signal processor 11, the DSC 12, the image processor 13, and the image memory 16 form an image producer 20.

The display controller 14 performs control such that the monitor 15 displays a three-dimensional ultrasound diagnostic image on the basis of three-dimensional image data input from the image processor 13.

The monitor 15 includes a display device, such as an LCD, and displays an ultrasound diagnostic image under the control of the display controller 14.

The apparatus controller 17 controls the respective units of the ultrasound diagnostic apparatus on the basis of a command input from the operating unit 18 by an operator. The apparatus controller 17 controls the transmission circuit 5 and the reception circuit 6 through the probe controller 7 such that either normal scan in which the transmission and reception of an ultrasonic beam is performed evenly over a space under observation including a region of interest or temperature rise suppressing scan in which the transmission and reception or the reception of an ultrasonic beam for at least a part of a region other than the region of interest in the spatial region under observation is paused is performed in accordance with the internal temperature Tp detected by the temperature sensor 8 of the ultrasound probe 1.

The operating unit 18 is configured to allow the operator to perform an input operation. The operating unit 18 constitutes a region of interest setter of the invention, and includes a keyboard, a mouse, a trackball, a touch panel, or the like.

The storage unit 19 stores an operation program or the like, and a recording medium, such as a hard disk, a flexible disk, an MO, an MT, a RAM, a CD-ROM, a DVD-ROM, an SD card, a CF card, or a USB memory, a server, or the like may be used.

The signal processor 11, the DSC 12, the image processor 13, the display controller 14, and the apparatus controller 17 are constituted by a CPU and operation programs for causing the CPU to perform various processes, and they may be constituted by digital circuits.

When producing a three-dimensional image, the transducer array 3 is electronically scanned by the transmission circuit 5 and the reception circuit 6, and transmits and receives an ultrasonic beam toward the subject to acquire two-dimensional image data in a single tomographic plane, and the transducer array 3 is mechanically scanned by the array moving unit 4 to collect two-dimensional image data corresponding to a large number of tomographic planes.

That is, ultrasonic waves are transmitted from a plurality of ultrasound transducers of the transducer array 3 in response to an actuation signal supplied from the transmission circuit 5 of the ultrasound probe 1, reception signals are output from the respective ultrasound transducers having received an ultrasonic echo from the subject to the reception circuit 6, and reception data is produced by the reception circuit 6. A B-mode image signal is produced by the signal processor 11 of the diagnostic apparatus body 2 to which reception data has been input, the B-mode image signal is raster-converted by the DSC 12, and various image processes are performed on the B-mode image signal in the image processor 13. Accordingly, two-dimensional image data in a single tomographic plane is produced and stored in the image memory 16.

In this way, while two-dimensional image data in a single tomographic plane is produced, the transducer array 3 is mechanically scanned by the array moving unit 4 with a predetermined angle range or stroke, such that two-dimensional image data corresponding to a large number of tomographic planes are sequentially produced and stored in the image memory 16. Three-dimensional image data for a space determined in the angle range or stroke of mechanical scan or the electronic scan range of the transducer array 3 is produced in the image processor 13 using image data stored in the image memory 16. A three-dimensional image is displayed on the monitor 15 by the display controller 14 on the basis of three-dimensional image data by an image projection method, such as VR (Volume Rendering) or MPR (Multiplanar Reconstruction).

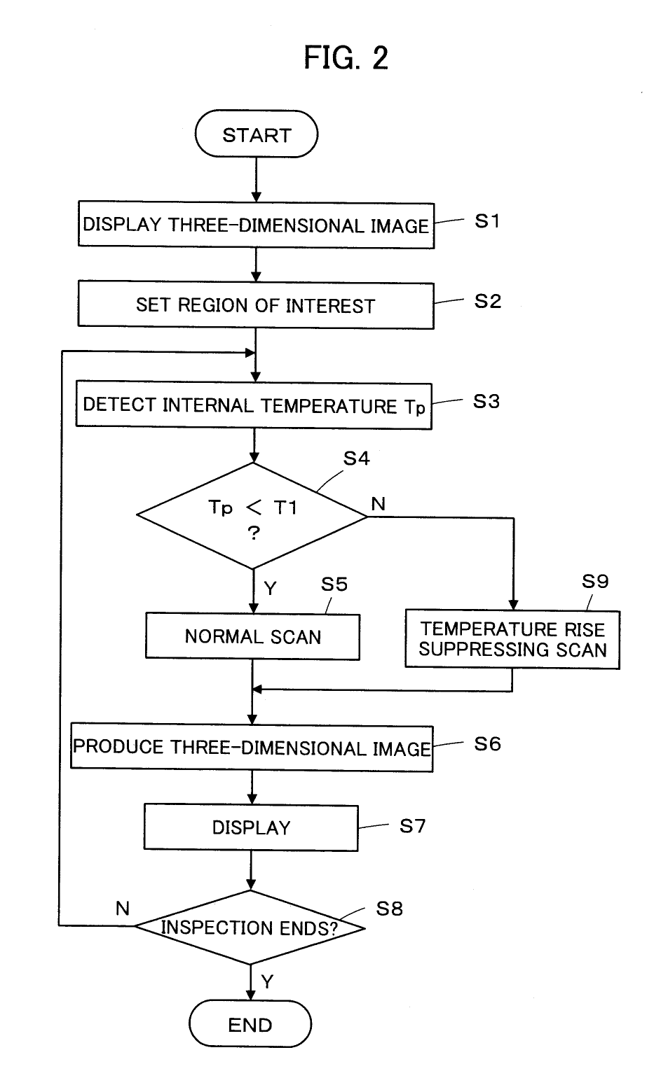

Next, the operation of Embodiment 1 will be described with reference to a flowchart of FIG. 2.

First, in Step S1, the transducer array 3 is electronically scanned by the transmission circuit 5 and the reception circuit 6 to acquire two-dimensional image data, and the transducer array 3 is mechanically scanned by the array moving unit 4 to produce three-dimensional image data. A three-dimensional image is displayed on the monitor 15 by the display controller 14.

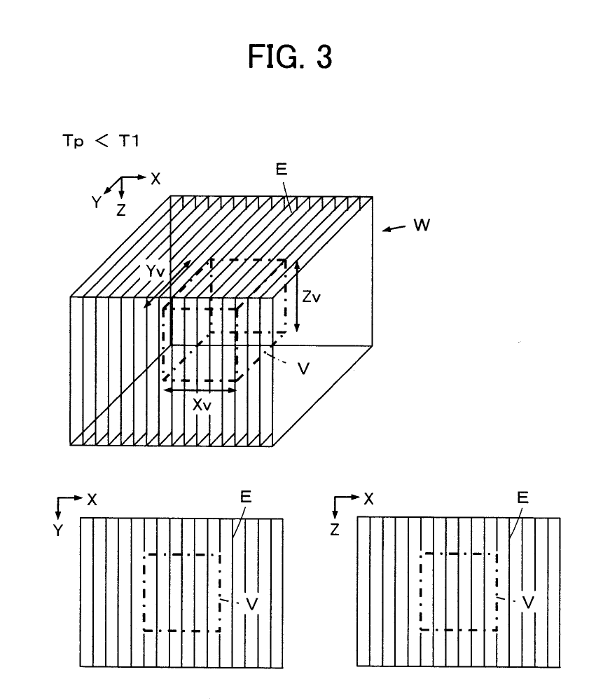

In Step S2, the operator operates the operating unit 18, and as shown in FIG. 3, a region V of interest is set on a three-dimensional image on a spatial region W under observation displayed on the monitor 15. In FIG. 3, the X axis represents a moving direction of the transducer array 3 by the array moving unit 4, that is, a mechanical scan direction, the Y axis represents a one-dimensional array direction of a plurality of ultrasound transducers of the transducer array 3, and the Z axis represents a measurement depth direction. It is assumed that the region V of interest has a size of Xv, Yv, and Zv in the X-axis direction, the Y-axis direction, and the Z-axis direction.

If the region V of interest is set, in Step S3, the internal temperature Tp of the ultrasound probe 1 is detected by the temperature sensor 8. In Step S4, the detected internal temperature Tp is compared with a first set value T1 set in advance.

When it is determined that the internal temperature Tp of the ultrasound probe 1 is lower than the first set value T1, the process progresses to Step S5, and the transmission circuit 5 and the reception circuit 6 are controlled by the apparatus controller 17 through the probe controller 7, and the normal scan is performed. That is, as shown in FIG. 3, the transducer array 3 is electronically scanned by the transmission circuit 5 and the reception circuit 6, and the transducer array 3 is mechanically scanned by the array moving unit 4. Therefore, electronic scan planes E are formed evenly over the spatial region W under observation, and two-dimensional image data for each electronic scan plane E is produced and stored in the image memory 16.

Next, in Step S6, three-dimensional image data for the spatial region W under observation is produced by the image processor 13 using two-dimensional image data stored in the image memory 16. Subsequently, in Step S7, a three-dimensional image is displayed on the monitor 15 by the display controller 14.

In Step S8, it is confirmed whether or not the inspection ends. While the inspection is continuing, Steps S3 to S8 are repeated. When the inspection ends, a sequence of processing is completed.

Ultrasound diagnosis is executed in the above-described manner, and as the execution time elapses, the internal temperature Tp of the ultrasound probe 1 gradually increases. Accordingly, in Step S4, when it is determined that the internal temperature Tp of the ultrasound probe 1 is equal to or higher than the first set value T1, the process progresses to Step S9, and the transmission circuit 5 and the reception circuit 6 are controlled by the apparatus controller 17 through the probe controller 7 such that the temperature rise suppressing scan is now performed.

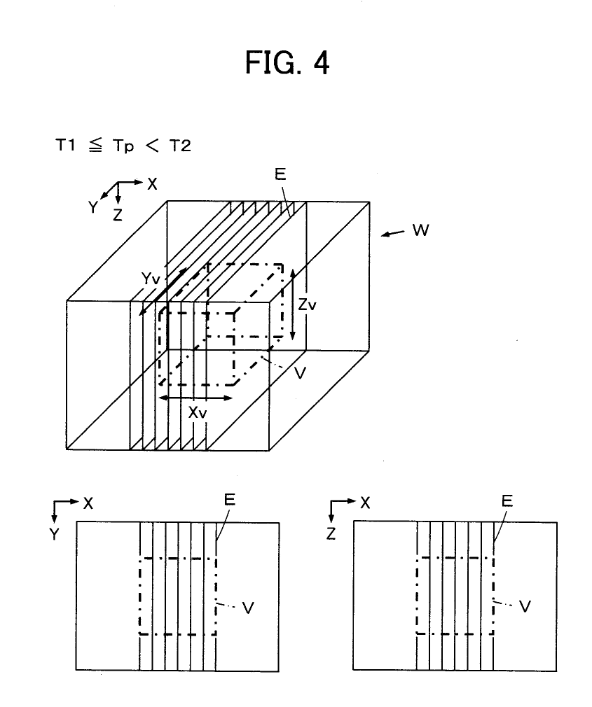

That is, as shown in FIG. 4, while the mechanical scan of the transducer array 3 by the array moving unit 4 is performed over the spatial region W under observation, regardless of the region V of interest, electronic scan plane E are formed only in a range of the length Xv including the region V of interest in the X-axis direction which is the mechanical scan direction of the transducer array 3, and the transmission and reception of an ultrasonic beam for a region other than the region V of interest in the X-axis direction is paused. The pause time of the transmission circuit 5 and the reception circuit 6 is extended by this amount, and a temperature rise in the ultrasound probe 1 is suppressed.

Thereafter, in Step S6, three-dimensional image data is produced using two-dimensional image data for each electronic scan plane E stored in the image memory 16 in the image processor 13, and in Step S7, a three-dimensional image is displayed on the monitor 15 by the display controller 14.

If the temperature rise suppressing scan is performed, and the internal temperature Tp of the ultrasound probe 1 decreases to be equal to or lower than the first set value T1, the normal scan is performed again, such that a three-dimensional image corresponding to the spatial region W under observation can be displayed.

As described above, when the internal temperature Tp of the ultrasound probe 1 detected by the temperature sensor 8 is equal to or higher than the first set value T1, the transmission circuit 5 and the reception circuit 6 are controlled such that the transmission and reception of an ultrasonic beam for a region other than the region V of interest in the mechanical scan direction of the transducer array 3 is paused. Therefore, it becomes possible to obtain a high-quality three-dimensional ultrasound image for at least the region V of interest while suppressing a rise in the internal temperature Tp of the ultrasound probe 1.

### Embodiment 2

Although in Embodiment 1 described above, the first set value T1 is set, and when the internal temperature Tp of the ultrasound probe 1 is equal to or higher than the first set value T1, the temperature rise suppressing scan is performed, a plurality of temperature set values may be set, and scan having different temperature rise suppressing effects may be performed in a stepwise manner depending on the internal temperature Tp of the ultrasound probe 1.

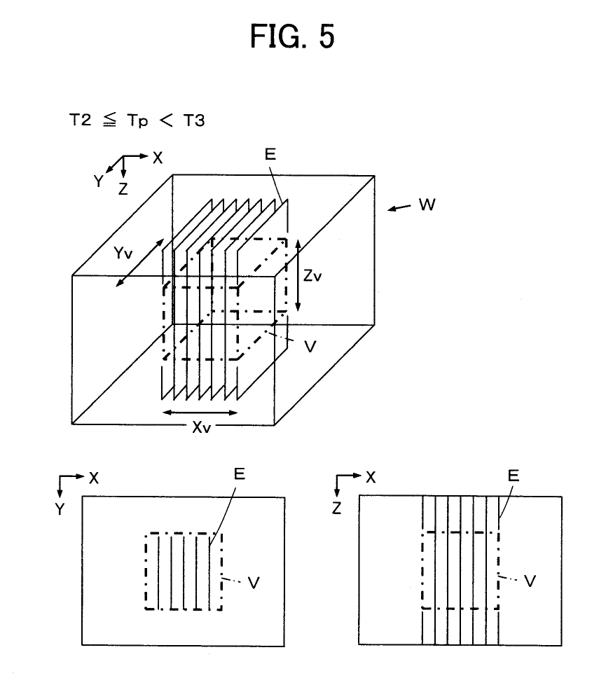

For example, a second set value T2 higher than the first set value T1 and a third set value T3 higher than the second set value T2 are set in advance, and when the internal temperature Tp of the ultrasound probe 1 detected by the temperature sensor 8 is equal to or higher than the first set value T1 and lower than the second set value T2, as shown in FIG. 4, in the mechanical scan direction of the transducer array 3, electronic scan planes E are formed only in a range of the length Xv including the region V of interest. When the internal temperature Tp of the ultrasound probe 1 is equal to or higher than the second set value T2 and lower than the third set value T3, as shown in FIG. 5, electronic scan planes E can be formed only in a range of the length Yv including the region V of interest in the Y-axis direction which is the one-dimensional array direction of the transducer array 3, and the transmission and reception of an ultrasonic beam for a region other than the region V of interest in the Y-axis direction can be paused.

When this happens, the range in which the transmission and reception of an ultrasonic beam is paused increases by the amount corresponding to the region other than the region V of interest in the Y-axis direction, and the pause period of the transmission circuit 5 and the reception circuit 6 is further extended by this amount, thereby suppressing a rise in the temperature of the ultrasound probe 1.

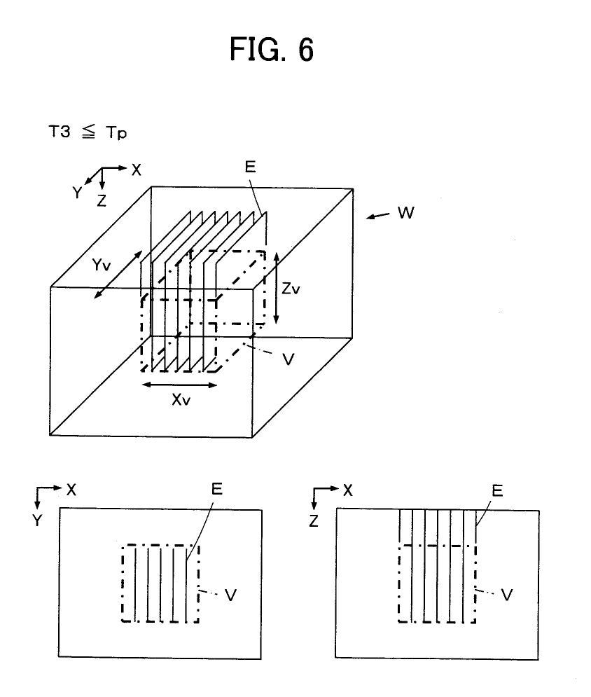

When the internal temperature Tp of the ultrasound probe 1 detected by the temperature sensor 8 is equal to or higher than the third set value T3, as shown in FIG. 6, electronic scan planes E can be formed only in a range of the length Zv including the region V of interest and a region shallower than the region V of interest in the Z-axis direction which is the measurement depth direction, and the reception of an ultrasonic beam for a region deeper than the region V of interest can be paused.

When this happens, the range in which the reception of an ultrasonic beam is paused increases by the amount corresponding to the region deeper than the region V of interest, and the pause period of the reception circuit 6 is further extended by this amount, thereby further suppressing a rise in the temperature of the ultrasound probe 1.

In Embodiment 2, the mechanical scan of the transducer array 3 by the array moving unit 4 is performed over the spatial region W under observation, regardless of the internal temperature Tp of the ultrasound probe 1 and the region V of interest.

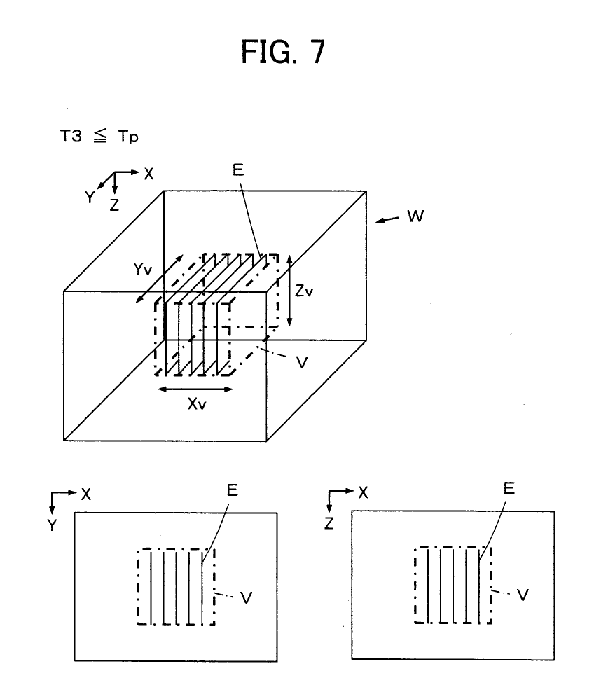

When the internal temperature Tp of the ultrasound probe 1 detected by the temperature sensor 8 is equal to or higher than the third set value T3, as shown in FIG. 7, electronic scan planes E may be formed only in a range of the length Zv including the region V of interest in the measurement depth direction, and the reception of an ultrasonic beam for a region other than the region V of interest in the Z-axis direction may be paused. It becomes possible to further extend the pause period of the reception circuit 6, compared to a case where the reception of an ultrasonic beam for a region shallower than the region V of interest is paused, as shown in FIG. 6.

### Embodiment 3

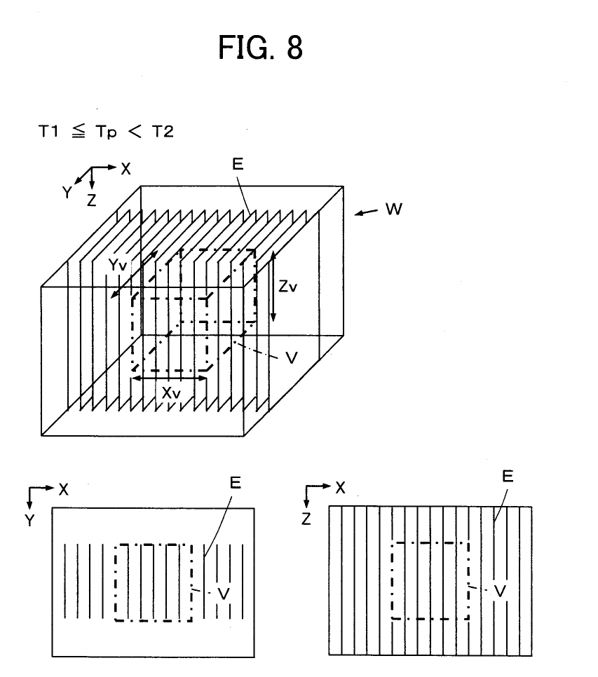

Although in Embodiment 1 described above, when the internal temperature Tp of the ultrasound probe 1 is equal to or higher than the first set value T1 and lower than the second set value T2, as shown in FIG. 4, the transmission and reception of an ultrasonic beam for a region other than the region V of interest in the X-axis direction which is the mechanical scan direction of the transducer array 3 is paused, the invention is not limited thereto. For example, as shown in FIG. 8, electronic scan planes E may be formed only in a range of the length Yv including the region V of interest in the Y-axis direction which is the one-dimensional array direction of the transducer array 3, and the transmission and reception of an ultrasonic beam for a region other than the region V of interest in the Y-axis direction may be paused.

In this case, a plurality of temperature set values are set, and when the internal temperature Tp of the ultrasound probe 1 increases to be equal to or higher than the second set value T2, the transmission and reception of an ultrasonic beam for a region other than the region V of interest in the X-axis direction which is the mechanical scan direction of the transducer array 3 may be paused or the reception of an ultrasonic beam for a region other than the region V of interest in the Z-axis direction which is the measurement depth direction may be paused.

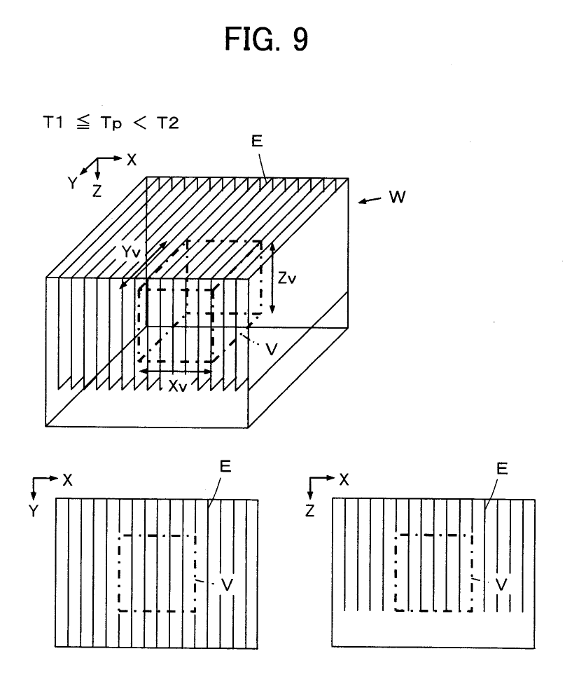

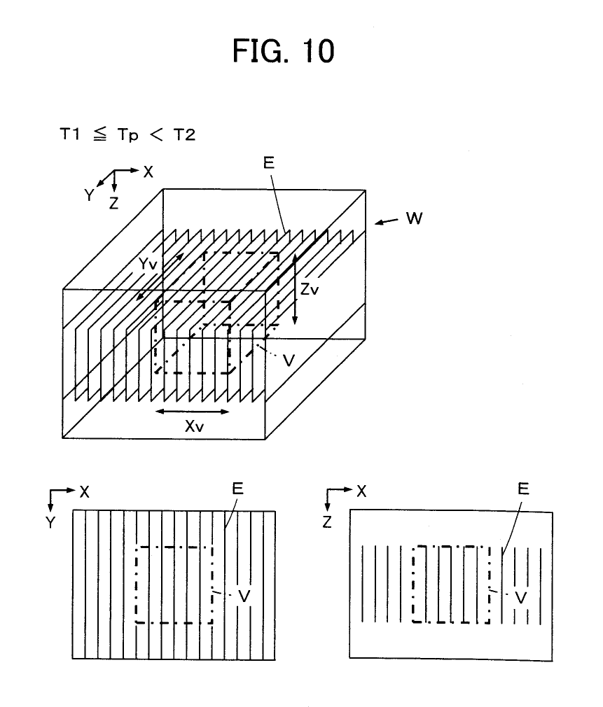

When the internal temperature Tp of the ultrasound probe 1 is equal to or higher than the first set value T1 and lower than the second set value T2, as shown in FIG. 9, electronic scan planes E may be formed only in a range of the length Zv including the region V of interest and a region shallower than the region V of interest in the Z-axis direction which is the measurement depth direction, and the reception of an ultrasonic beam for a region deeper than the region V of interest may be paused. Alternatively, as shown in FIG. 10, electronic scan planes E may be formed only in a range of the length Zv including the region V of interest in the Z-axis direction which is the measurement depth direction, and the reception of an ultrasonic beam for a region other than the region V of interest in the Z-axis direction may be paused.

Even when the scan shown in FIG. 9 or 10 is performed, a plurality of temperature set values may be set, and when the internal temperature Tp of the ultrasound probe 1 increases to be equal to or higher than the second set value T2, the transmission and reception of an ultrasonic beam for a region other than the region V of interest in the X-axis direction which is the mechanical scan direction of the transducer array 3 or for a region other than the region V of interest in the Y-axis direction which is the one-dimensional array direction of the transducer array 3 may be further paused.

In Embodiment 3, as in Embodiment 1, the pause period of the transmission circuit 5 and the reception circuit 6 or the pause period of the reception circuit 6 is extended, making it possible to obtain a high-quality three-dimensional ultrasound image for at least the region V of interest while suppressing a rise in the internal temperature Tp of the ultrasound probe 1.

### Embodiment 4

Although in Embodiments 1 to 3 described above, when the internal temperature Tp of the ultrasound probe 1 is equal to or higher than the first set value T1, the transmission and reception or the reception of an ultrasonic beam for at least a part of a region other than the region V of interest is paused, in Embodiment 4, the transmission and reception or the reception of an ultrasonic beam for at least a part of a region other than the region V of interest is intermittently performed.

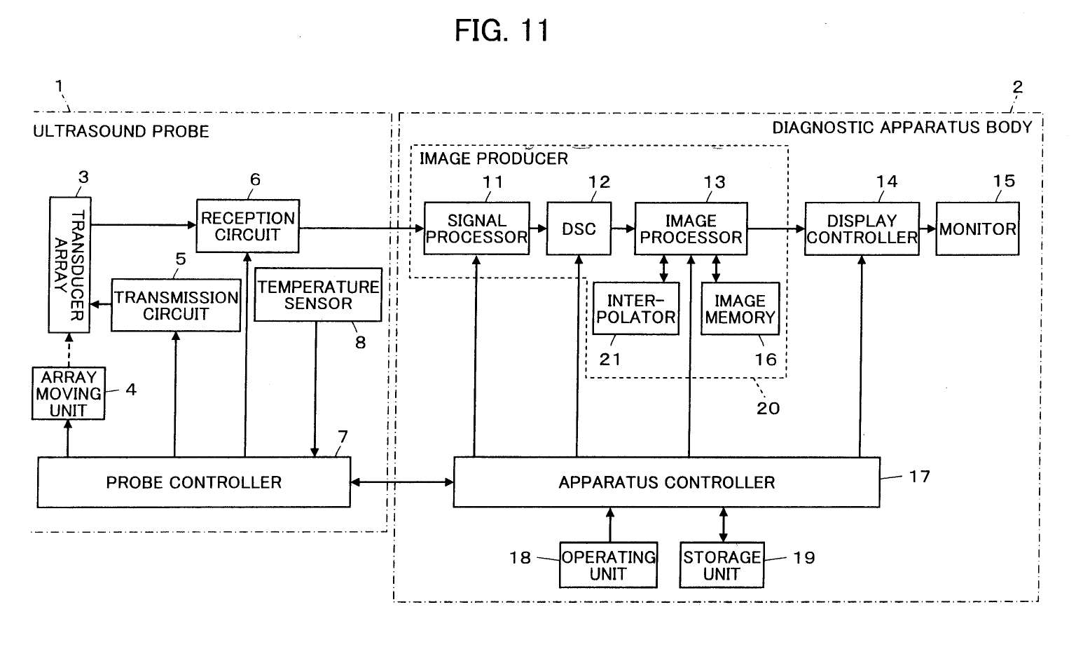

FIG. 11 shows the configuration of an ultrasound diagnostic apparatus according to Embodiment 4. The ultrasound diagnostic apparatus includes an ultrasound probe 1, and a diagnostic apparatus body 2A connected to the ultrasound probe 1.

The diagnostic apparatus body 2A is configured such that an interpolator 21 is connected to the image processor 13 in the diagnostic apparatus body 2 of Embodiment 1 shown in FIG. 1, and the apparatus controller 17 is connected to the interpolator 21.

The interpolator 21 interpolates and forms two-dimensional image data of an intermediate frame between previous and next frames on the basis of two-dimensional image data of the previous and next frames.

The signal processor 11, the DSC 12, the image processor 13, the image memory 16, and the interpolator 21 form an image producer 20A.

The apparatus controller 17 controls the transmission circuit 5 and the reception circuit 6 through the probe controller 7 such that either normal scan in which the transmission and reception of an ultrasonic beam is performed evenly over a spatial region under observation including a region of interest or temperature rise suppressing scan in which the transmission and reception or the reception of an ultrasonic beam for at least a part of a region other than the region of interest in the spatial region under observation is intermittently performed is performed in accordance with the internal temperature Tp detected by the temperature sensor 8 of the ultrasound probe 1.

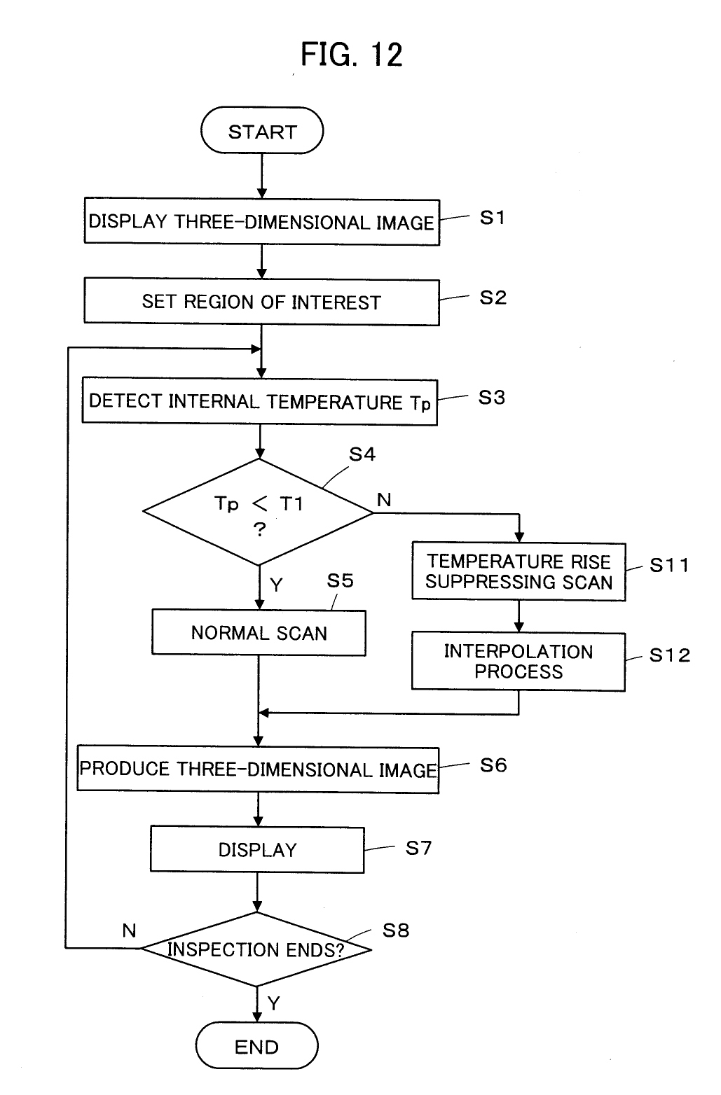

The operation of Embodiment 4 is shown in a flowchart of FIG. 12. Steps S1 to S8 are the same as the operation in embodiment 1 shown in FIG. 2. That is, when the internal temperature Tp of the ultrasound probe 1 is lower than the first set value T1, the same normal scan as in Embodiment 1 is performed.

In Step S4, when it is determined that the internal temperature Tp of the ultrasound probe 1 is equal to or higher than the first set value T1, the process progresses to Step S11, and the transmission circuit 5 and the reception circuit 6 are controlled by the apparatus controller 17 through the probe controller 7 such that the temperature rise suppressing scan is performed.

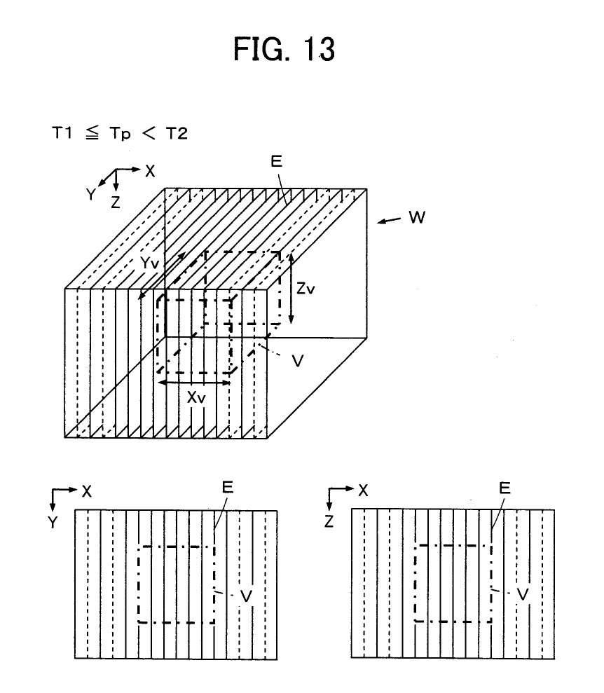

At this time, as shown in FIG. 13, while the mechanical scan of the transducer array 3 by the array moving unit 4 is performed over the spatial region W under observation regardless of the region V of interest, electronic scan planes E are formed evenly in a range of the length Xv including the region V of interest in the X-axis direction which is the mechanical scan direction of the transducer array 3 as in the normal scan, and the transmission and reception of an ultrasonic beam for a region other than the region V of interest in the X-axis direction is intermittently performed frame by frame. In FIG. 13, the formed electronic scan plane E is indicated by a solid line, and an electronic scan plane being not formed is indicated by a dotted line.

For this reason, while electronic scan planes E are formed in the range including the region V of interest in the X-axis direction at the same interval as in the normal scan, the number of electronic scan planes E out of the range is reduced compared to the normal scan, and the interval between the electronic scan planes E being formed is expanded. The pause period of the transmission circuit 5 and the reception circuit 6 is extended by the amount corresponding to the electronic scan planes having not been formed compared to the normal scan, thereby suppressing a rise in the temperature of the ultrasound probe 1.

If the temperature rise suppressing scan is performed in the above-described manner, and two-dimensional image data for each formed electronic scan plane E is stored in the image memory 16, in Step S12, an interpolation process of two-dimensional image data is performed by the interpolator 21. That is, two-dimensional image data of a frame where the transmission and reception of an ultrasonic beam has not been performed and no electronic scan plane has been formed in a region other than the region V of interest in the X-axis direction is interpolated and formed on the basis of two-dimensional image data of the previous and next frames.

Accordingly, two-dimensional image data of the same number of frames as when the normal scan is performed is produced, and in Step S6, the image processor 13 produces three-dimensional image data using two-dimensional image data. Subsequently, in Step S7, a three-dimensional image is displayed on the monitor 15 by the display controller 14.

If the temperature rise suppressing scan is performed, and the internal temperature Tp of the ultrasound probe 1 decreases to be equal to or lower than the first set value T1, the normal scan is performed again, and a three-dimensional image can be displayed.

As described above, when the internal temperature Tp of the ultrasound probe 1 detected by the temperature sensor 8 is equal to or higher than the first set value T1, the transmission circuit 5 and the reception circuit 6 are controlled such that the transmission and reception of an ultrasonic beam for a region other than the region V of interest in the mechanical scan direction of the transducer array 3 is intermittently performed. Therefore, it becomes possible to obtain a high-quality three-dimensional ultrasound image for at least the region V of interest while suppressing a rise in the internal temperature Tp of the ultrasound probe 1.

### Embodiment 5

Although in Embodiment 4 described above, the first set value T1 is set, and when the internal temperature Tp of the ultrasound probe 1 is equal to or higher than the first set value T1, the temperature rise suppressing scan is performed, a plurality of temperature set values may be set, and scan having different temperature rise suppressing effects may be performed in a stepwise manner depending on the internal temperature Tp of the ultrasound probe 1.

For example, a second set value T2 higher than the first set value T1 and a third set value T3 higher than the second set value T2 are set in advance, and when the internal temperature Tp of the ultrasound probe 1 detected by the temperature sensor 8 is equal to or higher than the first set value T1 and lower than the second set value T2, as shown in FIG. 13, the transmission and reception of an ultrasonic beam is performed in a range of the length Xv including the region V of interest in the X-axis direction which is the mechanical scan direction of the transducer array 3 as in the normal scan, and the transmission and reception of an ultrasonic beam for a region other than the region V of interest is intermittently performed.

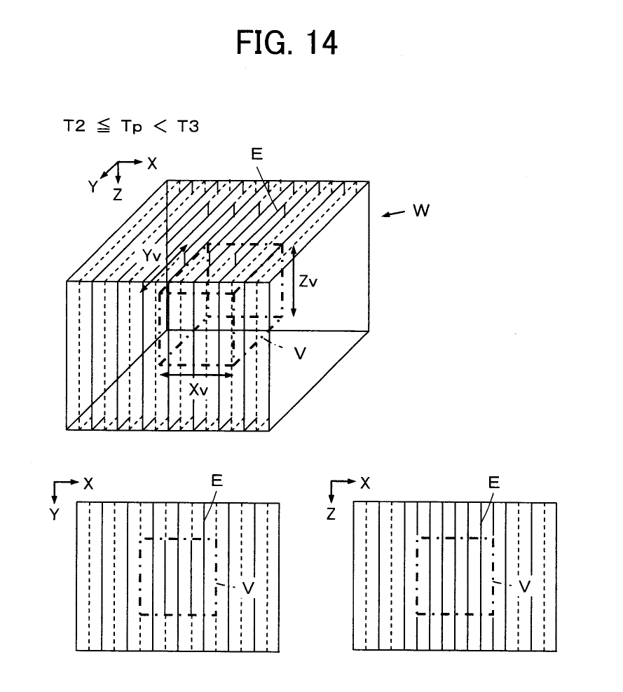

When the internal temperature Tp of the ultrasound probe 1 is equal to or higher than the second set value T2 and lower than the third set value T3, as shown in FIG. 14, the transmission and reception of an ultrasonic beam can be further performed in a range of the length Yv including the region V of interest in the Y-axis direction which is the one-dimensional array direction of the transducer array 3 as in the normal scan, and the transmission and reception of an ultrasonic beam for a region other than the region V of interest in the Y-axis direction can be intermittently performed.

When this happens, the range in which the transmission and reception of an ultrasonic beam is not performed increases by an amount such that the transmission and reception of an ultrasonic beam is intermittently performed for a region other than the region V of interest in the Y-axis direction, and the pause period of the transmission circuit 5 and the reception circuit 6 is further extended by this amount, thereby suppressing a rise in the temperature of the ultrasound probe 1.

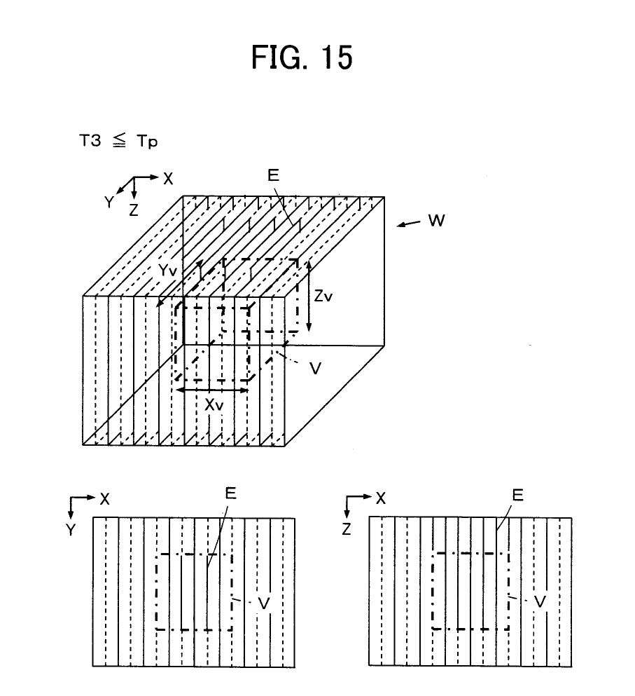

When the internal temperature Tp of the ultrasound probe 1 detected by the temperature sensor 8 is equal to or higher than the third set value T3, as shown in FIG. 15, the transmission and reception of an ultrasonic beam may be further performed only in a range of the length Zv including the region V of interest and a region shallower than the region V of interest in the Z-axis direction which is the measurement depth direction as in the normal scan, and the reception of an ultrasonic beam for a region deeper than the region V of interest may be intermittently performed.

When this happens, the range in which the reception of an ultrasonic beam is intermittently performed increases by the amount corresponding to the region deeper than the region V of interest, and the pause period of the reception circuit 6 is further extended by this amount, thereby further suppressing a rise in the temperature of the ultrasound probe 1.

In Embodiment 5, the mechanical scan of the transducer array 3 by the array moving unit 4 is performed over the spatial region W under observation regardless of the internal temperature Tp of the ultrasound probe 1 and the region V of interest.

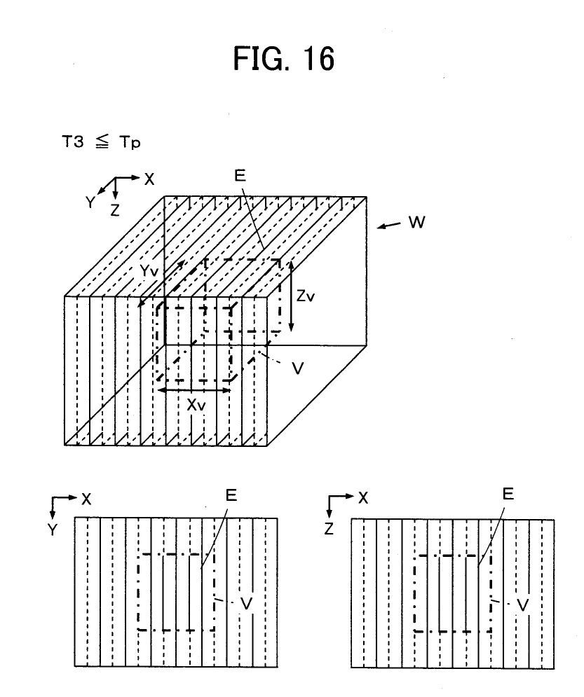

When the internal temperature Tp of the ultrasound probe 1 detected by the temperature sensor 8 is equal to or higher than the third set value T3, as shown in FIG. 16, the transmission and reception of an ultrasonic beam may be performed only in a range of the length Zv including the region V of interest in the measurement depth direction as in the normal scan, and the reception of an ultrasonic beam for a region other than the region V of interest in the Z-axis direction may be intermittently performed. It becomes possible to further extend the pause period of the reception circuit 6 compared to a case where the reception of an ultrasonic beam for a region deeper than the region V of interest is intermittently performed as shown in FIG. 15.

### Embodiment 6

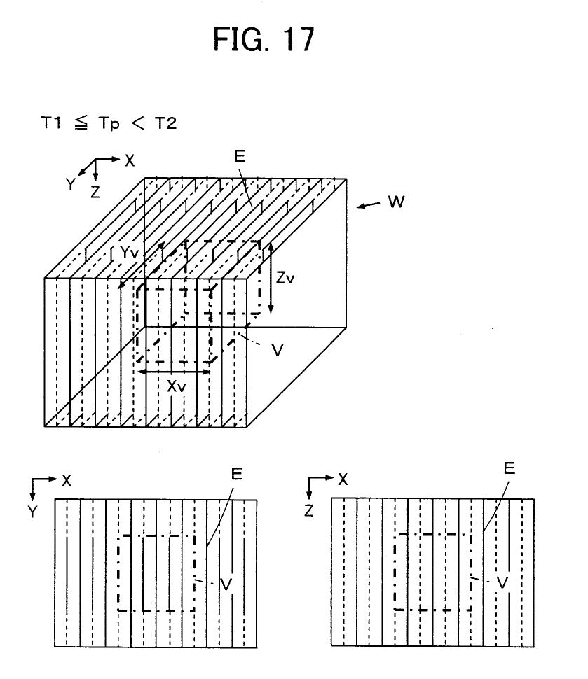

Although in Embodiment 4 described above, when the internal temperature Tp of the ultrasound probe 1 is equal to or higher than the first set value T1 and lower than the second set value T2, as shown in FIG. 13, the transmission and reception of an ultrasonic beam for a region other than the region V of interest in the X-axis direction which is the mechanical scan direction of the transducer array 3 is intermittently performed, the invention is not limited thereto. For example, as shown in FIG. 17, the transmission and reception of an ultrasonic beam may be performed only in a range of the length Yv including the region V of interest in the Y-axis direction which is the one-dimensional array direction of the transducer array 3 as in the normal scan, and the transmission and reception of an ultrasonic beam for a region other than the region V of interest in the Y-axis direction may be intermittently performed.

In this case, a plurality of temperature set values are set, and when the internal temperature Tp of the ultrasound probe 1 increases to be equal to or higher than the second set value T2, the transmission and reception of an ultrasonic beam for a region other than the region V of interest in the X-axis direction which is the mechanical scan direction of the transducer array 3 may be further intermittently performed, or the reception of an ultrasonic beam for a region other than the region V of interest in the Z-axis direction which is the measurement depth direction may be intermittently performed.

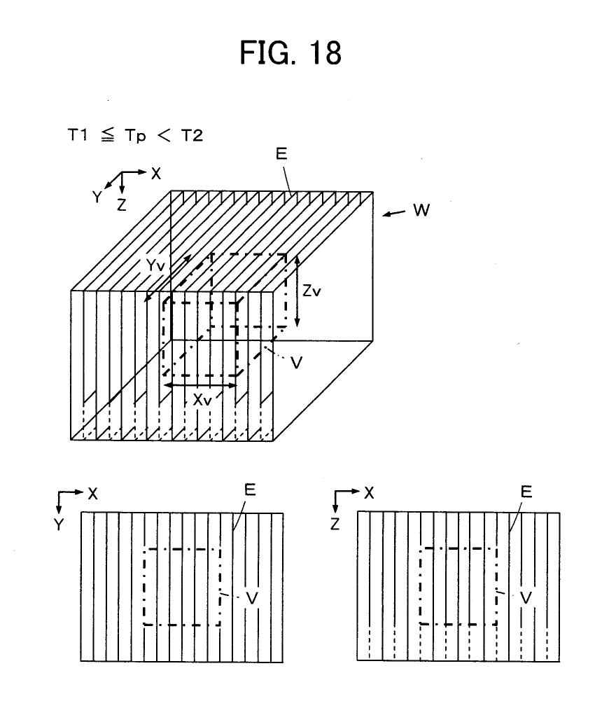

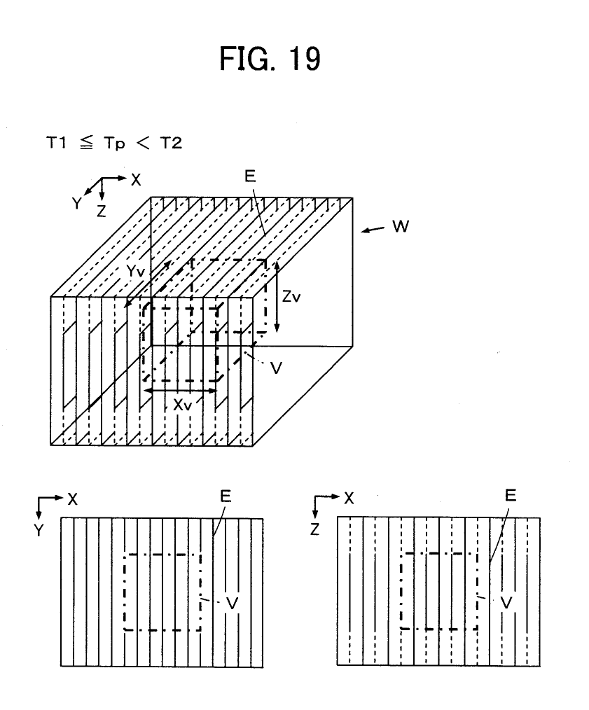

When the internal temperature Tp of the ultrasound probe 1 is equal to or higher than the first set value T1 and lower than the second set value T2, as shown in FIG. 18, the transmission and reception of an ultrasonic beam may be performed only in a range of the length Zv including the region V of interest and a region shallower than the region V of interest in the Z-axis direction which is the measurement depth direction as in the normal scan, and the reception of an ultrasonic beam for a region deeper than the region V of interest may be intermittently performed. Alternatively, as shown in FIG. 19, the transmission and reception of an ultrasonic beam may be performed only in a range of the length Zv including the region V of interest in the Z-axis direction which is the measurement depth direction as in the normal scan, and the reception of an ultrasonic beam for a region other than the region V of interest in the Z-axis direction may be intermittently performed.

Even when the scan shown in FIG. 18 or 19 is performed, a plurality of temperature set values may be set, and when the internal temperature Tp of the ultrasound probe 1 increases to be equal to or higher than the second set value T2, the transmission and reception of an ultrasonic beam for a region other than the region V of interest in the X-axis direction which is the mechanical scan direction of the transducer array 3 or a region other than the region V of interest in the Y-axis direction which is the one-dimensional array direction of the transducer array 3 may be further intermittently performed.

In Embodiment 6, as in Embodiment 4, the pause period of the transmission circuit 5 and the reception circuit 6 or the pause period of the reception circuit 6 is extended, making it possible to obtain a high-quality three-dimensional ultrasound image for at least the region V of interest while suppressing a rise in the internal temperature Tp of the ultrasound probe 1.

### Embodiment 7

Although in Embodiments 4 to 6 described above, when the internal temperature Tp of the ultrasound probe 1 is equal to or higher than the first set value T1, the transmission and reception or the reception of an ultrasonic beam for at least a part of a region other than the region V of interest is intermittently performed, in Embodiment 7, the transmission and reception or the reception of an ultrasonic beam for at least a part of a region other than the region V of interest is performed with decreased spatial resolution.

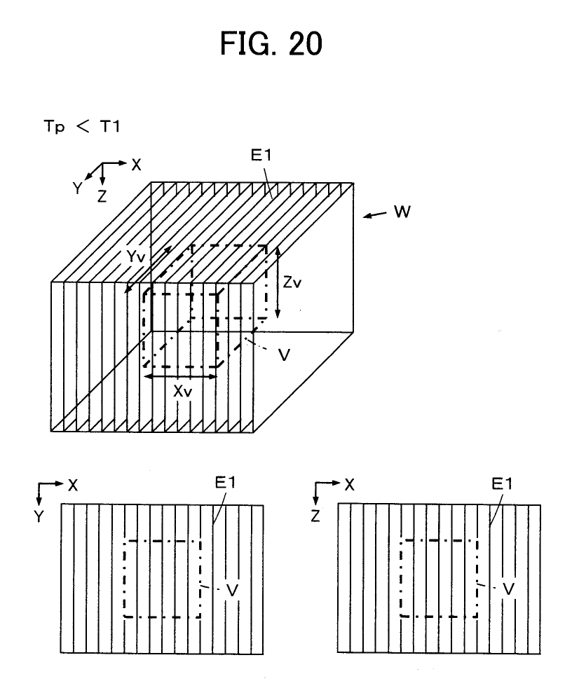

An ultrasound diagnostic apparatus of Embodiment 7 has the same configuration as the ultrasound diagnostic apparatus of Embodiment 1 shown in FIG. 1. When the internal temperature Tp of the ultrasound probe 1 is lower than the first set value T1, the same normal scan as in Embodiment 1 is performed. That is, as shown in FIG. 20, while the transducer array 3 is electronically scanned by the transmission circuit 5 and the reception circuit 6, the transducer array 3 is mechanically scanned by the array moving unit 4, such that electronic scan planes E1 are formed over the spatial region W under observation, and two-dimensional image data for each electronic scan plane E1 is produced and stored in the image memory 16. In the normal scan, it is assumed that the reception of an ultrasonic beam is performed using a predetermined number N of simultaneous opening channels, and a predetermined number S of sound rays per frame are formed.

Ultrasound diagnosis is performed in the above-described manner, and when it is determined that the internal temperature Tp of the ultrasound probe 1 is equal to or higher than the first set value T1, the transmission circuit 5 and the reception circuit 6 are controlled by the apparatus controller 17 through the probe controller 7 such that the temperature rise suppressing scan is now performed.

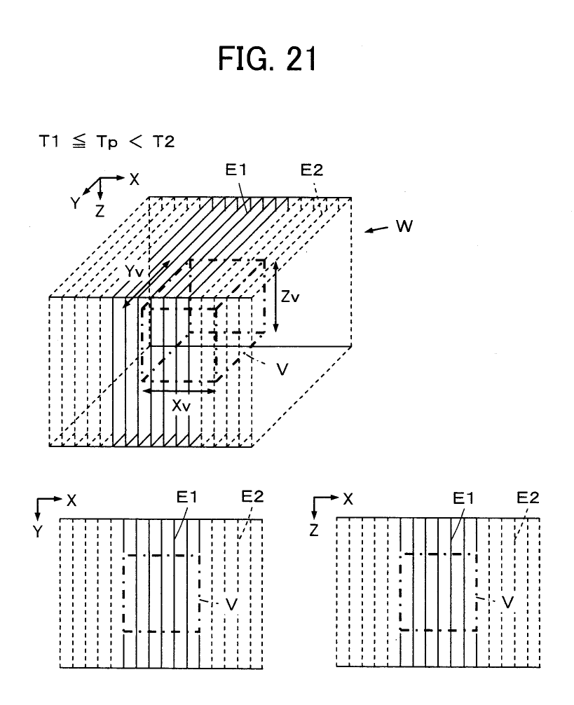

That is, as shown in FIG. 21, while the mechanical scan of the transducer array 3 by the array moving unit 4 is performed over the spatial region W under observation regardless of the region V of interest, the transducer array 3 is electronically scanned only in a range of the length Xv including the region V of interest in the X-axis direction which is the mechanical scan direction of the transducer array 3 as in the normal scan so as to form S sound rays per frame with the number N of simultaneous opening channels at the time of the reception. Thus, electronic scan planes E1 are formed. The transducer array 3 is electronically scanned for a region other than the region V of interest in the X-axis direction the number of sound rays per frame or the number of simultaneous opening channels at the time of the reception is reduced compared to the normal scan. Thus, electronic scan planes E2 are formed. In FIG. 21, each electronic scan plane E1 which is formed by the same number N of simultaneous opening channels at the time of the reception and the same number S of sound rays per frame as in the normal scan is indicated by a solid line. Each electronic scan plane E2 which is formed by the temperature rise suppressing scan where the number of sound rays per frame or the number of simultaneous opening channels at the time of the reception is reduced compared to the normal scan is indicated by a dotted line.

With the reduction in the number of sound rays per frame or the reduction in the number of simultaneous opening channels at the time of the reception, the spatial resolution decreases, and image quality for a region other than the region V of interest in the X-axis direction is degraded. Meanwhile, the pause period of the transmission circuit 5 and the reception circuit 6 is extended by this amount, thereby suppressing a rise in the temperature of the ultrasound probe 1.

If two-dimensional image data for each of the electronic scan planes E1 and E2 formed in the above-described manner is stored in the image memory 16, the image processor 13 produces three-dimensional image data using two-dimensional image data, and a three-dimensional image is displayed on the monitor 15 by the display controller 14.

If the temperature rise suppressing scan is performed, and the internal temperature Tp of the ultrasound probe 1 decreases to be equal to or lower than the first set value T1, the normal scan is performed again, and a three-dimensional image can be displayed.

As described above, when the internal temperature Tp of the ultrasound probe 1 detected by the temperature sensor 8 is equal to or higher than the first set value T1, the transmission circuit 5 and the reception circuit 6 are controlled such that, for a region other than the region V of interest in the mechanical scan direction of the transducer array 3, the number of sound rays per frame or the number of simultaneous opening channels at the time of the reception is reduced to decrease spatial resolution. Therefore, it becomes possible to obtain a high-quality three-dimensional ultrasound image for at least the region V of interest while suppressing a rise in the internal temperature Tp of the ultrasound probe 1.

### Embodiment 8

Although in Embodiment 7 described above, the first set value T1 is set, and when the internal temperature Tp of the ultrasound probe 1 is equal to or higher than the first set value T1, the temperature rise suppressing scan is performed, a plurality of temperature set values may be set, and scan having different temperature rise suppressing effects may be performed in a stepwise manner depending on the internal temperature Tp of the ultrasound probe 1.

For example, a second set value T2 higher than the first set value T1 and a third set value T3 higher than the second set value T2 are set in advance, and when the internal temperature Tp of the ultrasound probe 1 detected by the temperature sensor 8 is equal to or higher than the first set value T1 and lower than the second set value T2, as shown in FIG. 21, the transmission and reception of an ultrasonic beam is performed such that spatial resolution becomes equal to that in the normal scan in a range of the length Xv including the region V of interest in the X-axis direction which is the mechanical scan direction of the transducer array 3. For a region other than the region V of interest, the transmission and reception of an ultrasonic beam is performed with decreased spatial resolution.

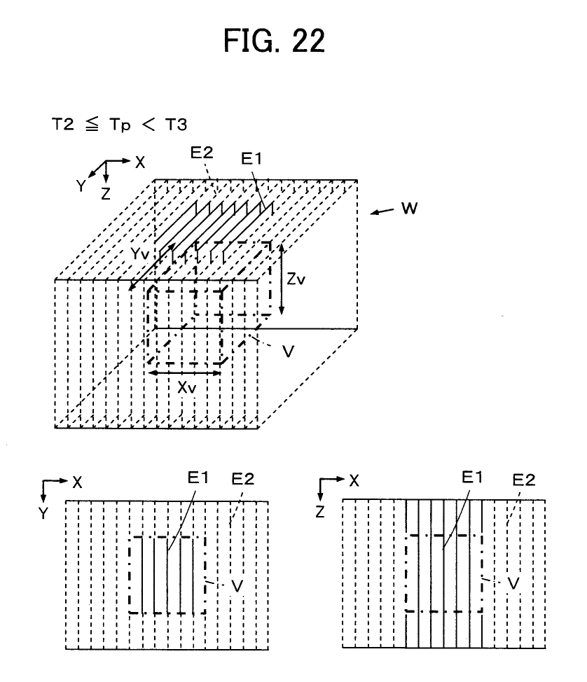

When the internal temperature Tp of the ultrasound probe 1 is equal to or higher than the second set value T2 and lower than the third set value T3, as shown in FIG. 22, the transmission and reception of an ultrasonic beam can be further performed such that spatial resolution becomes equal to that in the normal scan in a range of the length Yv including the region V of interest in the Y-axis direction which is the one-dimensional array direction of the transducer array 3. For a region other than the region V of interest in the Y-axis direction, the transmission and reception of an ultrasonic beam can be performed with decreased spatial resolution.

When this happens, the pause period of the transmission circuit 5 and the reception circuit 6 is further extended by an amount such that the transmission and reception of an ultrasonic beam for a region other than the region V of interest in the Y-axis direction is performed with decreased spatial resolution, thereby suppressing a rise in the temperature of the ultrasound probe 1.

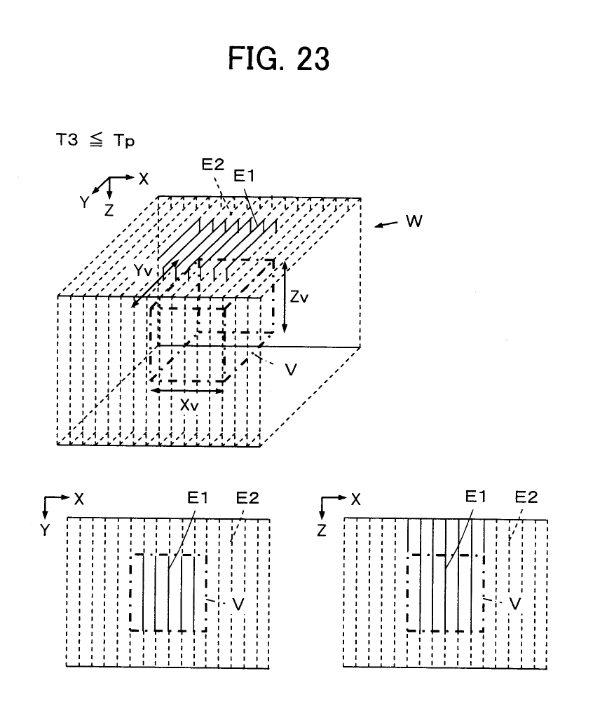

When the internal temperature Tp of the ultrasound probe 1 detected by the temperature sensor 8 is equal to or higher than the third set value T3, as shown in FIG. 23, the transmission and reception of an ultrasonic beam may be performed such that spatial resolution becomes equal to that in the normal scan only in a range of the length Zv including the region V of interest and a region shallower than the region V of interest in the Z-axis direction which is the measurement depth direction. For a region deeper than the region V of interest, the reception of an ultrasonic beam may be performed with decreased spatial resolution.

When this happens, the range in which the reception of an ultrasonic beam is performed with decreased spatial resolution increases by the amount corresponding to the region deeper than the region V of interest, and the pause period of the reception circuit 6 is further extended by this amount, thereby further suppressing a rise in the temperature of the ultrasound probe 1.

In Embodiment 8, the mechanical scan of the transducer array 3 by the array moving unit 4 is performed over the spatial region W under observation regardless of the internal temperature Tp of the ultrasound probe 1 and the region V of interest.

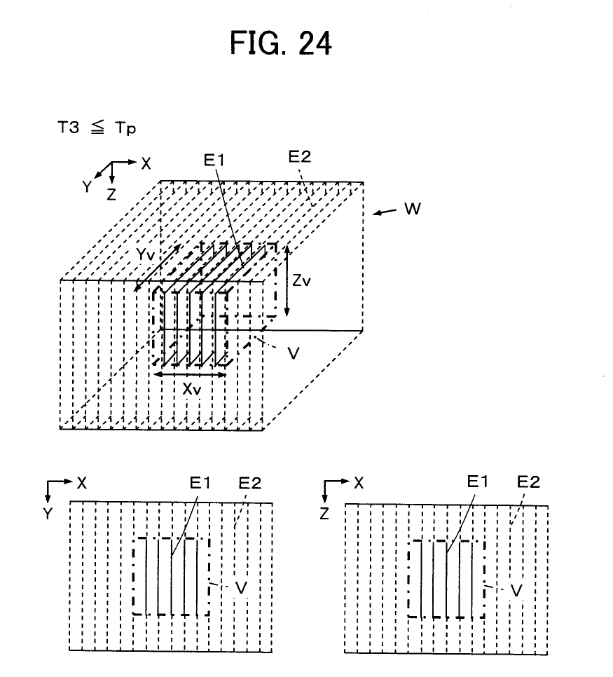

When the internal temperature Tp of the ultrasound probe 1 detected by the temperature sensor 8 is equal to or higher than the third set value T3, as shown in FIG. 24, the transmission and reception of an ultrasonic beam may be performed such that spatial resolution becomes equal to that in the normal scan only in a range of the length Zv including the region V of interest in the measurement depth direction. For a region other than the region V of interest in the Z-axis direction, the reception of an ultrasonic beam may be performed with decreased spatial resolution. It becomes possible to further extend the pause period of the reception circuit 6 compared to a case where the reception of an ultrasonic beam for a region deeper than the region V of interest is performed with low spatial resolution as shown in FIG. 23.

### Embodiment 9

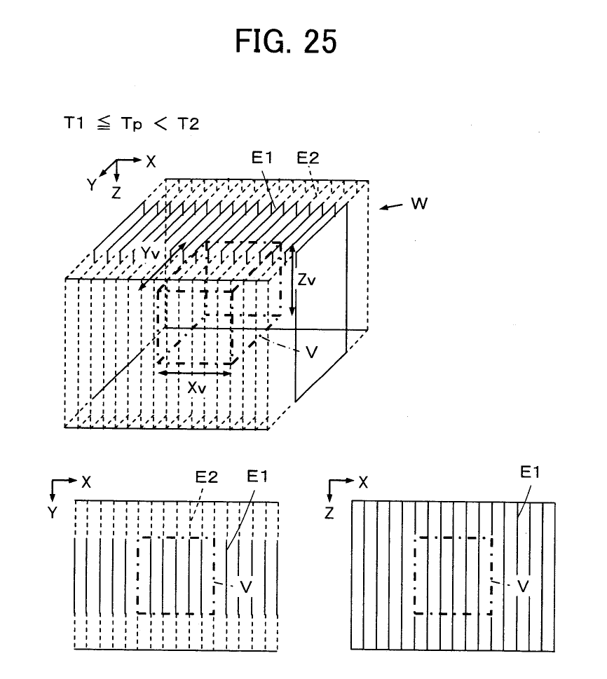

Although in Embodiment 7 described above, when the internal temperature Tp of the ultrasound probe 1 is equal to or higher than the first set value T1 and lower than the second set value T2, as shown in FIG. 21, for a region other than the region V of interest in the X-axis direction which is the mechanical scan direction of the transducer array 3, the transmission and reception of an ultrasonic beam is performed with decreased spatial resolution, the invention is not limited thereto. For example, as shown in FIG. 25, the transmission and reception of an ultrasonic beam may be performed such that spatial resolution becomes equal to that in the normal scan only in a range of the length Yv including the region V of interest in the Y-axis direction which is the one-dimensional array direction of the transducer array 3. For a region other than the region V of interest in the Y-axis direction, the transmission and reception of an ultrasonic beam may be performed with decreased spatial resolution.

In this case, a plurality of temperature set values are set, and when the internal temperature Tp of the ultrasound probe 1 increases to be equal to or higher than the second set value T2, for a region other than the region V of interest in the X-axis direction which is the mechanical scan direction of the transducer array 3 or a region other than the region V of interest in the Z-axis direction which is the measurement depth direction, the reception of an ultrasonic beam may be performed with decreased spatial resolution.

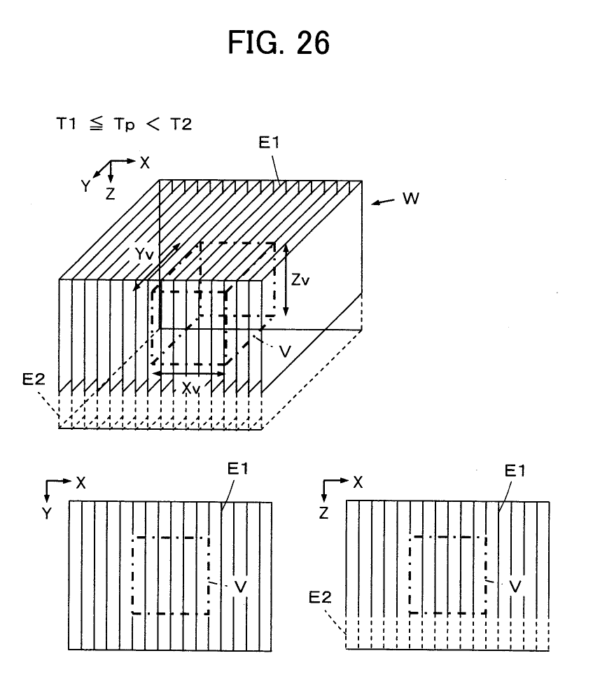

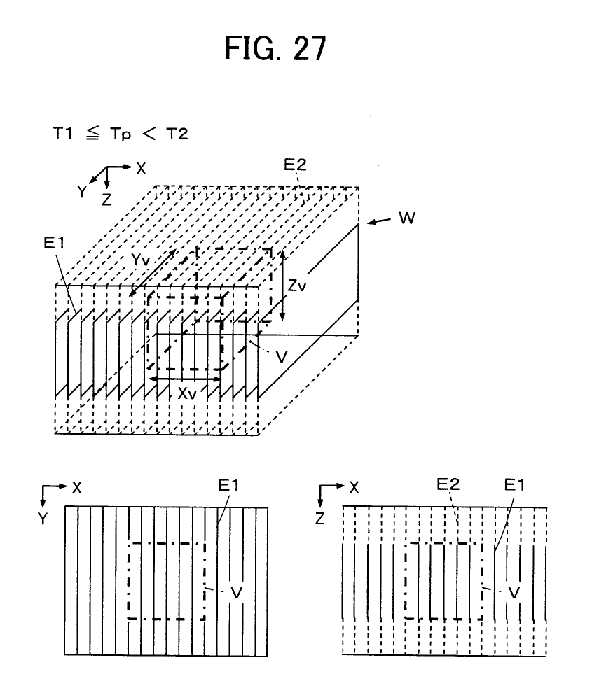

When the internal temperature Tp of the ultrasound probe 1 is equal to or higher than the first set value T1 and lower than the second set value T2, as shown in FIG. 26, the transmission and reception of an ultrasonic beam may be performed such that spatial resolution becomes equal to that in the normal scan only in a range of the length Zv including the region V of interest and a region shallower than the region V of interest in the Z-axis direction which is the measurement depth direction. For a region deeper than the region V of interest, the reception of an ultrasonic beam may be performed with decreased spatial resolution. Alternatively, as shown in FIG. 27, the transmission and reception of an ultrasonic beam may be performed such that spatial resolution becomes equal to that in the normal scan only in a range of the length Zv including the region V of interest in the Z-axis direction which is the measurement depth direction. For a region other than the region V of interest in the Z-axis direction, the reception of an ultrasonic beam may be performed with decreased spatial resolution.

Even when the scan shown in FIG. 26 or 27 is performed, a plurality of temperature set values may be set, when the internal temperature Tp of the ultrasound probe 1 increases to be equal to or higher than the second set value T2, for a region other than the region V of interest in the X-axis direction which is the mechanical scan direction of the transducer array 3 or a region other than the region V of interest in the Y-axis direction which is the one-dimensional array direction of the transducer array 3, the transmission and reception of an ultrasonic beam may be performed with decreased spatial resolution.

In Embodiment 9, as in Embodiment 7, the pause period of the transmission circuit 5 and the reception circuit 6 or the pause period of the reception circuit 6 is extended, making it possible to obtain a high-quality three-dimensional ultrasound image for at least the region V of interest while suppressing a rise in the internal temperature Tp of the ultrasound probe 1.

The connection of the ultrasound probe 1 and the diagnostic apparatus body 2 in Embodiments 1 to 9 described above may be either wired connection or connection by wireless communication.

What is claimed is:

1. An ultrasound diagnostic apparatus comprising:
an ultrasound probe which has a one-dimensional array-type transducer array and an array moving unit moving the transducer array in a direction substantially orthogonal to the array direction of the transducer array;
a transmission and reception circuit which electronically scans with the transducer array, and transmits and receives an ultrasonic beam toward a subject to acquire two-dimensional image data;
an image producer which produces a three-dimensional ultrasound image using two-dimensional image data acquired by the transmission and reception circuit while mechanically scanning with the transducer array in a direction substantially orthogonal to the array direction of the transducer array by the array moving unit;
a region of interest setter which sets a region of interest in an imaging region;
a temperature sensor which detects an internal temperature of the ultrasound probe; and
a controller which, when the internal temperature of the ultrasound probe detected by the temperature sensor is equal to or higher than a first set value, controls the transmission and reception circuit such that the transmission and reception or the reception of an ultrasonic beam for at least a part of a region other than the region of interest set by the region of interest setter is paused.

2. The ultrasound diagnostic apparatus according to claim 1,
wherein, when the internal temperature of the ultrasound probe detected by the temperature sensor is equal to or higher than the first set value, the controller controls the transmission and reception circuit such that the transmission and reception of an ultrasonic beam for a region other than the region of interest in a mechanical scan direction of the transducer array is paused.

3. The ultrasound diagnostic apparatus according to claim 2,
wherein, when the internal temperature of the ultrasound probe detected by the temperature sensor is equal to or higher than a second set value which is set to be higher than the first set value, the controller further controls the transmission and reception circuit such that the transmission and reception of an ultrasonic beam for a region other than the region of interest in the array direction of the transducer array is paused.

4. The ultrasound diagnostic apparatus according to claim 3,
wherein, when the internal temperature of the ultrasound probe detected by the temperature sensor is equal to or higher than a third set value which is set to be higher than the second set value, the controller further controls the transmission and reception circuit such that the reception of an ultrasonic beam for a region deeper than the region of interest is paused.

5. The ultrasound diagnostic apparatus according to claim 3,
wherein, when the internal temperature of the ultrasound probe detected by the temperature sensor is equal to or higher than a third set value which is set to be higher than the second set value, the controller further controls the transmission and reception circuit such that the reception of an ultrasonic beam for a region other than the region of interest in a measurement depth direction is paused.

6. The ultrasound diagnostic apparatus according to claim 1,
wherein, when the internal temperature of the ultrasound probe detected by the temperature sensor is equal to or higher than the first set value, the controller controls the transmission and reception circuit such that the transmission and reception of an ultrasonic beam for a region other than the region of interest in the array direction of the transducer array is paused.

7. The ultrasound diagnostic apparatus according to claim 1,
wherein, when the internal temperature of the ultrasound probe detected by the temperature sensor is equal to or higher than the first set value, the controller controls the transmission and reception circuit such that the reception of an ultrasonic beam for a region deeper than the region of interest is paused.

8. The ultrasound diagnostic apparatus according to claim 1,
wherein, when the internal temperature of the ultrasound probe detected by the temperature sensor is equal to or higher than the first set value, the controller controls the transmission and reception circuit such that the reception of an ultrasonic beam for a region other than the region of interest in a measurement depth direction is paused.

9. An ultrasound diagnostic apparatus comprising:
an ultrasound probe which has a one-dimensional array-type transducer array and an array moving unit moving the transducer array in a direction substantially orthogonal to the array direction of the transducer array;
a transmission and reception circuit which electronically scans with the transducer array, and transmits and receives an ultrasonic beam toward a subject to acquire two-dimensional image data;
an image producer which produces a three-dimensional ultrasound image using two-dimensional image data acquired by the transmission and reception circuit while mechanically scanning with the transducer array in a direction substantially orthogonal to the array direction of the transducer array by the array moving unit;
a region of interest setter which sets a region of interest in an imaging region;
a temperature sensor which detects an internal temperature of the ultrasound probe; and
a controller which, when the internal temperature of the ultrasound probe detected by the temperature sensor is equal to or higher than a first set value, controls the transmission and reception circuit such that the transmission and reception or the reception of an ultrasonic beam for at least a part of a region other than the region of interest set by the region of interest setter is intermittently performed.

10. The ultrasound diagnostic apparatus according to claim 9, further comprising:
an interpolator which interpolates and forms two-dimensional image data of an intermediate frame on the basis of two-dimensional image data of previous and next frames,
wherein, when the internal temperature of the ultrasound probe detected by the temperature sensor is equal to or higher than the first set value, the controller controls the transmission and reception circuit such that the transmission and reception or the reception of an ultrasonic beam for at least a part of a region other than the region of interest is intermittently performed frame by frame, and
two-dimensional image data of a frame where the transmission and reception of an ultrasonic beam for at least a part of a region other than the region of interest has not been performed is interpolated and formed by the interpolator.

11. The ultrasound diagnostic apparatus according to claim 9,
wherein, when the internal temperature of the ultrasound probe detected by the temperature sensor is equal to or higher than the first set value, the controller controls the transmission and reception circuit such that the transmission and reception of an ultrasonic beam for a region other than the region of interest in a mechanical scan direction of the transducer array is intermittently performed.

12. The ultrasound diagnostic apparatus according to claim 11,
wherein, when the internal temperature of the ultrasound probe detected by the temperature sensor is equal to or higher than a second set value which is set to be higher than the first set value, the controller further controls the transmission and reception circuit such that the transmission and reception of an ultrasonic beam for a region other than the region of interest in the array direction of the transducer array is intermittently performed.

13. The ultrasound diagnostic apparatus according to claim 12,
wherein, when the internal temperature of the ultrasound probe detected by the temperature sensor is equal to or higher than a third set value which is set to be higher than the second set value, the controller further controls the transmission and reception circuit such that the reception of an ultrasonic beam for a region deeper than the region of interest is intermittently performed.

14. The ultrasound diagnostic apparatus according to claim 12,
wherein, when the internal temperature of the ultrasound probe detected by the temperature sensor is equal to or higher than a third set value which is set to be higher than the second set value, the controller further controls the transmission and reception circuit such that the reception of an ultrasonic beam for a region other than the region of interest in a measurement depth direction is intermittently performed.

15. The ultrasound diagnostic apparatus according to claim 9,
wherein, when the internal temperature of the ultrasound probe detected by the temperature sensor is equal to or higher than the first set value, the controller controls the transmission and reception circuit such that the transmission and reception of an ultrasonic beam for a region other than the region of interest in the array direction of the transducer array is intermittently performed.

16. The ultrasound diagnostic apparatus according to claim 9,
wherein, when the internal temperature of the ultrasound probe detected by the temperature sensor is equal to or higher than the first set value, the controller controls the transmission and reception circuit such that the reception of an ultrasonic beam for a region deeper than the region of interest is intermittently performed.

17. The ultrasound diagnostic apparatus according to claim 9,
wherein, when the internal temperature of the ultrasound probe detected by the temperature sensor is equal to or higher than the first set value, the controller controls the transmission and reception circuit such that the reception of an ultrasonic beam for a region other than the region of interest in a measurement depth direction is intermittently performed.

18. An ultrasound diagnostic apparatus comprising:
an ultrasound probe which has a one-dimensional array-type transducer array and an array moving unit moving the transducer array in a direction substantially orthogonal to the array direction of the transducer array;
a transmission and reception circuit which electronically scans with the transducer array, and transmits and receives an ultrasonic beam toward a subject to acquire two-dimensional image data;
an image producer which produces a three-dimensional ultrasound image using two-dimensional image data acquired by the transmission and reception circuit while mechanically scanning with the transducer array in a direction substantially orthogonal to the array direction of the transducer array by the array moving unit;
a region of interest setter which sets a region of interest in an imaging region;
a temperature sensor which detects an internal temperature of the ultrasound probe; and
a controller which, when the internal temperature of the ultrasound probe detected by the temperature sensor is equal to or higher than a first set value, controls the transmission and reception circuit such that the transmission and reception or the reception of an ultrasonic beam for at least a part of a region other than the region of interest set by the region of interest setter is performed with decreased spatial resolution.

19. The ultrasound diagnostic apparatus according to claim 18,
wherein the controller reduces the number of sound rays per frame or reduces the number of simultaneous opening channels at the time of reception to form the decreased spatial resolution.

20. The ultrasound diagnostic apparatus according to claim 18,
wherein, when the internal temperature of the ultrasound probe detected by the temperature sensor is equal to or higher than the first set value, the controller controls the transmission and reception circuit such that the transmission and reception of an ultrasonic beam for a region other than the region of interest in a mechanical scan direction of the transducer array is performed with the decreased spatial resolution.

21. The ultrasound diagnostic apparatus according to claim 20,
wherein, when the internal temperature of the ultrasound probe detected by the temperature sensor is equal to or higher than a second set value which is set to be higher than the first set value, the controller further controls the transmission and reception circuit such that the transmission and reception of an ultrasonic beam for a region other than the region of interest in the array direction of the transducer array is performed with the decreased spatial resolution.

22. The ultrasound diagnostic apparatus according to claim 21,
wherein, when the internal temperature of the ultrasound probe detected by the temperature sensor is equal to or higher than a third set value which is set to be higher than the second set value, the controller further controls the transmission and reception circuit such that the reception of an ultrasonic beam for a region deeper than the region of interest is performed with the decreased spatial resolution.

23. The ultrasound diagnostic apparatus according to claim 21,
wherein, when the internal temperature of the ultrasound probe detected by the temperature sensor is equal to or higher than a third set value which is set to be higher than the second set value, the controller further controls the transmission and reception circuit such that the reception of an ultrasonic beam for a region other than the region of interest in a measurement depth direction is performed with the decreased spatial resolution.

24. The ultrasound diagnostic apparatus according to claim 18,
wherein, when the internal temperature of the ultrasound probe detected by the temperature sensor is equal to or higher than the first set value, the controller controls the transmission and reception circuit such that the transmission and reception of an ultrasonic beam for a region other than the region of interest in the array direction of the transducer array is performed with the decreased spatial resolution.

25. The ultrasound diagnostic apparatus according to claim 18,
wherein, when the internal temperature of the ultrasound probe detected by the temperature sensor is equal to or higher than the first set value, the controller controls the transmission and reception circuit such that the reception of an ultrasonic beam for a region deeper than the region of interest is performed with the decreased spatial resolution.

26. The ultrasound diagnostic apparatus according to claim 18,
wherein, when the internal temperature of the ultrasound probe detected by the temperature sensor is equal to or higher than the first set value, the controller controls the transmission and reception circuit such that the reception of an ultrasonic beam for a region other than the region of interest in a measurement depth direction is performed with the decreased spatial resolution.

27. A method of producing an ultrasound image, the method comprising the steps of:
electronically scanning with a one-dimensional array-type transducer array of an ultrasound probe by a transmission and reception circuit and transmitting and receiving an ultrasonic beam toward a subject to acquire two-dimensional image data, and mechanically scanning with the transducer array in a direction substantially orthogonal to the array direction of the transducer array to acquire a plurality of pieces of two-dimensional image data;
producing a three-dimensional ultrasound image using a plurality of pieces of acquired two-dimensional image data;
setting a region of interest in an imaging region;
detecting an internal temperature of the ultrasound probe; and
when the detected internal temperature of the ultrasound probe is equal to or higher than a first set value, controlling the transmission and reception circuit such that the transmission and reception or the reception of an ultrasonic beam for at least a part of a region other than the region of interest is paused.

28. A method of producing an ultrasound image, the method comprising the steps of:
electronically scanning with a one-dimensional array-type transducer array of an ultrasound probe by a transmission and reception circuit and transmitting and receiving an ultrasonic beam toward a subject to acquire two-dimensional image data, and mechanically scanning with the transducer array in a direction substantially orthogonal to the array direction of the transducer array to acquire a plurality of pieces of two-dimensional image data;
producing a three-dimensional ultrasound image using a plurality of pieces of acquired two-dimensional image data;
setting a region of interest in an imaging region;
detecting an internal temperature of the ultrasound probe; and
when the detected internal temperature of the ultrasound probe is equal to or higher than a first set value, controlling the transmission and reception circuit such that the transmission and reception or the reception of an ultrasonic beam for at least a part of a region other than the region of interest is intermittently performed.

29. A method of producing an ultrasound image, the method comprising the steps of:
electronically scanning with a one-dimensional array-type transducer array of an ultrasound probe by a transmission and reception circuit and transmitting and receiving an ultrasonic beam toward a subject to acquire two-dimensional image data, and mechanically scanning with the transducer array in a direction substantially orthogonal to the array direction of the transducer array to acquire a plurality of pieces of two-dimensional image data;
producing a three-dimensional ultrasound image using a plurality of pieces of acquired two-dimensional image data;
setting a region of interest in an imaging region;
detecting an internal temperature of the ultrasound probe; and
when the detected internal temperature of the ultrasound probe is equal to or higher than a first set value, controlling the transmission and reception circuit such that the transmission and reception or the reception of an ultrasonic beam for at least a part of a region other than the region of interest is performed with decreased spatial resolution.
